SEQUENCE LISTING (6 sequences)

SEQ ID NO: 1 - DNA, 10 bp, Artificial Sequence
  Primer for use in Random Amplified Polymorphic DNA
ggtgcgggaa

SEQ ID NO: 2 - DNA, 10 bp, Artificial Seqence
  Primer for Random Amplified Polymorphic DNA
gtttcgctcc

SEQ ID NO: 3 - DNA, 10 bp, Artificial Sequence
  Primer for Random Amplified Polymorphic DNA
gtagacccgt

SEQ ID NO: 4 - DNA, 10 bp, Artificial Sequence
  Primer for Random Amplified Polymorphic DNA
aagagcccgt

SEQ ID NO: 5 - DNA, 10 bp, Artificial Sequence
  Primer for Random Amplified Polymorphic DNA
aacgcgcaac

SEQ ID NO: 6 - DNA, 10 bp, Artificial Sequence
  Primer for Random Amplified Polymorphic DNA
cccgtcagca

(19) United States
(12) Patent Application Publication
(10) Pub. No.: US 2012/0276058 A1
(43) Pub. Date: Nov. 1, 2012

(54) METHANE REDUCING STRAINS AND METHODS OF USE

(75) Inventors: Alexandra Helena Smith, Greendale, WI (US); Thomas G. Rehberger, Wauwatosa, WI (US)

(73) Assignee: DANISCO A/S, Copenhagen (DK)

(21) Appl. No.: 13/456,741

(22) Filed: Apr. 26, 2012

Related U.S. Application Data
(60) Provisional application No. 61/479,155, filed on Apr. 26, 2011.

(51) Int. Cl. A61K 35/74; A61P 3/00; A61P 1/14; C12N 1/20

(52) U.S. Cl. 424/93.4; 435/252.1

(57) ABSTRACT

Strains and compositions of bacterium of the genus Propionibacterium are provided that reduce methane production in a ruminant animal. Strains and compositions can be used as a feed supplement. Also provided are methods for reducing methane production in a ruminant animal comprising the step of administering to the ruminant animal an effective amount of at least one strain of bacterium of the genus Propionibacterium.

### CROSS-REFERENCE TO RELATED APPLICATIONS

This application claims priority under 35 U.S.C. §119(e) to U.S. Provisional Patent Application No. 61/479,155 filed Apr. 26, 2011, the entirety of which is incorporated by reference herein.

### BIBLIOGRAPHY

Complete bibliographic citations of the references referred to herein by the first author's last name in parentheses can be found in the Bibliography section, immediately preceding the claims.

### FIELD

The disclosure relates to bacterial strains and compositions that reduce methane production in an animal and methods of making and using those strains and compositions.

### BACKGROUND

Methane is a potent greenhouse gas implicated in global warming. Between 1750 and 1998 atmospheric methane increased by 149% from 700 to 1745 parts per billion. It is estimated that 55-70% of methane emissions are from anthropogenic activities and that 20-25% of these emissions are from ruminant eructation (Thorpe, 2009). There is a strong relationship between the increase in atmospheric methane concentrations and global ruminant populations. Not only does methanogenesis in ruminants increase greenhouse gas emissions, but it is also energetically wasteful to the animal resulting in a loss of 2 to 12% ingested feed energy. Therefore reducing methanogenesis in ruminants will not only reduce greenhouse gas emissions, but also increase feed efficiency in the animal.

### SUMMARY

Strains, methods and compositions for reducing methane production in a ruminant animal are provided. In one embodiment, a method for reducing methane production in a ruminant animal is provided comprising administering to the ruminant animal an effective amount of at least one strain of bacterium of the genus Propionibacterium.

In yet another embodiment, a feed supplement for a ruminant animal is disclosed. In still another embodiment, the feed supplement reduces methane production. In yet another embodiment, the feed supplement comprises at least one strain of bacterium of the genus Propionibacterium.

In another embodiment, a feed for a ruminant animal is disclosed. In still another embodiment, the feed reduces methane production. In yet another embodiment, the feed is supplemented with at least one strain of bacterium of the genus Propionibacterium.

In yet another embodiment, a method for reducing methane production is disclosed. In one embodiment, the method comprises administering to the animal a feed supplement comprising an effective amount of at least one strain of bacterium of the genus Propionibacterium. In still another embodiment, the method further comprises increasing feed efficiency or propionate production in the ruminant. In yet another embodiment, the method further comprises increasing feed efficiency and propionate production in the ruminant.

In another embodiment, the strain of bacterium belongs to the species Propionibacterium jensenii, Propionibacterium acidipropionici, Propionibacterium freudenreichii or Propionibacterium freudenreichii ssp shermanii. In yet another embodiment, the strain is selected from the group consisting of P169, P170, P179, P195, P261, P5, P63, P54, P25, P49, P104, strains having all the characteristics thereof, any derivative or variant thereof, and mixtures thereof.

In another embodiment, the effective amount of at least one strain of bacterium is administered to the ruminant animal by supplementing food intended for the animal with an effective amount of at least one strain of bacterium. In still another embodiment, the ruminant animal is selected from the members of the Ruminantia and Tylopoda suborders.

### BRIEF DESCRIPTION OF THE DRAWINGS

FIG. 1 is a diagram showing fermentation pathways within the rumen with hydrogen-consuming and producing reactions highlighted.

Before explaining embodiments of the invention in detail, it is to be understood that the invention is not limited in its application to the details of construction and the arrangement of the components set forth in the following description or illustrated in the drawings. The invention is capable of other embodiments or being practiced or carried out in various ways. Also, it is to be understood that the phraseology and terminology employed herein is for the purpose of description and should not be regarded as limiting.

### DETAILED DESCRIPTION

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, melt index, temperature etc., is from 100 to 1,000, it is intended that all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, relative amounts of components in a mixture, and various temperature and other parameter ranges recited herein.

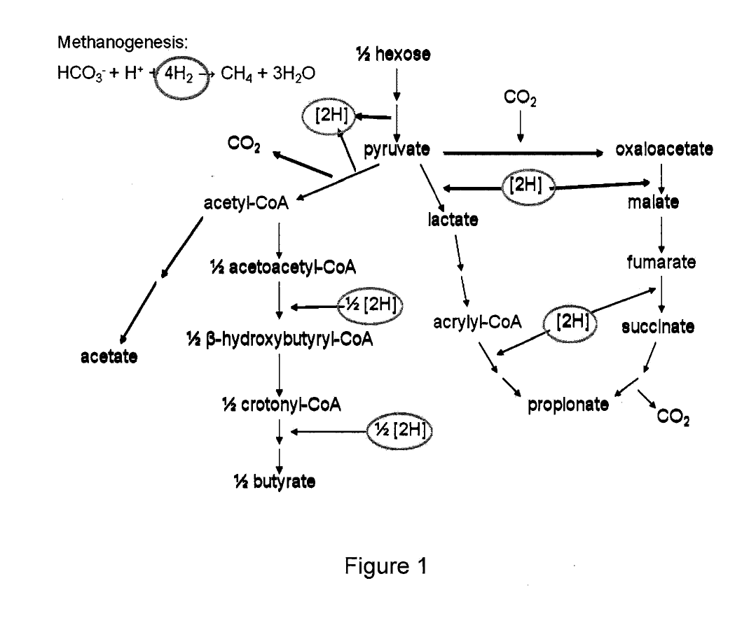

The production of propionate is a hydrogen-consuming reaction. The fermentation of pyruvate to propionate uses hydrogen at two points in the fermentation pathways, as is shown in FIG. 1. The inventors found that the production of propionate decreases the hydrogen partial pressure in the rumen. The inventors also found strains of bacterium of the genus Propionibacterium with an affinity for hydrogen utilization and propionate production. When these strains are fed to ruminants, the strains act as an alternative hydrogen sink, thereby reducing methane levels produced by the rumen and thus, the ruminants.

Strains and methods for reducing methane production in a ruminant animal are provided. The method comprises the step of administering to the ruminant animal an effective amount of at least one strain of bacterium of the genus Propionibacterium.

In at least some embodiments, the strain of bacterium belongs to the species Propionibacterium jensenii, Propionibacterium acidipropionici, Propionibacterium freudenreichii or Propionibacterium freudenreichii ssp shermanii.

Strains that can be used to reduce methane in ruminants include Propionibacterium strains P169, P170, P179, P195, P261, P5, P63, P54, P25, P49, and P104.

The P169 and P170 strains are available from the microorganism collection of the American Type Culture Collection (ATCC), 10801 University Blvd., Manassas, Va. 20110, under accession numbers ATCC PTA-5271 and ATCC PTA-5272, respectively, and were deposited on Jun. 18, 2003. Strains P179, P195, and P261 were deposited on Apr. 2, 2008 at the Agricultural Research Service Culture Collection (NRRL), 1815 North University Street. Peoria, Ill., 61604 and given accession numbers NRRL B-50133, NRRL B-50132, and NRRL B-50131, respectively. U.S. Pat. Nos. 6,951,643 and 7,470,531 describe strains P169, P170, P179, P195 and P261. The disclosures of U.S. Pat. Nos. 6,951,643 and 7,470,531 are incorporated herein by reference.

Propionibacteria jensenii strain P63 was deposited on Jan. 15, 2009, in the Deutsche Sammlung von Mikroorganismen and Zellkulturen GmbH (DSMZ, Inhoffenstr. 7 B, D-38124 Braunschweig, Germany) under number DSM22192 by Danisco Deutschland GmbH (Bush-Johannsen-Str. 1, 25899 Niebüll, Germany).

Strain P5 was deposited on Aug. 31, 1993 at the American Type Culture Collection (ATCC), 10801 University Blvd., Manassas, Va. 20110, under accession number ATCC 55467. Strain P5 is described in U.S. Pat. Nos. 6,120,810 and 6,221,650. The disclosures of U.S. Pat. Nos. 6,120,810 and 6,221,650 are incorporated herein by reference.

Strains P54, P25, P49, and P104 were deposited on Apr. 21, 2011 at the Agricultural Research Service Culture Collection (NRRL), 1815 North University Street, Peoria, Ill., 61604. Accession number NRRL B-50494 was given to P54, accession number NRRL B-50495 was given to P104, accession number NRRL B-50496 was given to P49, and accession number NRRL B-50497 was given to P25.

Methods of growing the strains are described in U.S. Pat. Nos. 6,951,643 and 7,470,531.

All deposits were done under the Budapest Treaty.

The inventors have found that Propionibacterium acidipropionici, freudenreichii and jensenii strains P169, P170, P179, P195, P5, P63, P54, P25, P49, and P104 numerically reduced the methane accumulation in vitro. Propionibacterium strain P261 is believed to also reduce methane accumulation in vitro.

Strains having all the characteristics of Propionibacterium strains P169, P170, P179, P195, P261, P5, P63, P54, P25, P49, and P104 are also included and are useful in the methods and compositions described and claimed herein.

Any derivative or variant of Propionibacterium strains P169, P170, P179, P195, P261, P5, P63, P54, P25, P49, and P104 are also included and are useful in the methods and compositions described and claimed herein.

In some embodiments, an effective amount of at least one strain of bacterium is administered to the ruminant animal by supplementing food intended for the animal with an effective amount of at least one strain of bacterium.

The ruminant animal can be selected from the members of the Ruminantia and Tylopoda suborders.

The ruminant animal can be selected from the members of the Antilocapridae, Bovidae, Cervidae, Giraffidae, Moschidae, Tragulidae families.

The ruminant animal can be a cattle, goat, sheep, giraffe, bison, yak, water buffalo, deer, camel, alpaca, llama, wildebeest, antelope, pronghorn or nilgai.

In some embodiments, the ruminant animal is a cattle or sheep. In at least some embodiments, the ruminant animal is a cattle.

A feed supplement for a ruminant animal for reducing methane production is provided comprising at least one strain of bacterium the genus Propionibacterium.

A feed for a ruminant animal is also provided, wherein the feed is supplemented with a feed supplement described herein.

Surprisingly and unexpectedly, the inventors have shown that certain bacteria possess the property of reducing methane production in ruminant animals. These bacteria belong to the genus Propionibacterium. Also provided herein is a method for reducing methane production in a ruminant animal comprising the step of administering to the ruminant animal an effective amount of at least one strain of bacterium of the genus Propionibacterium.

A ruminant is a mammal of the order Artiodactyla that digests plant-based food by initially softening it within the animal's first stomach, then regurgitating the semi-digested mass, now known as cud, and chewing it again. The process of rechewing the cud to further break down plant matter and stimulate digestion is called “ruminating.” Ruminants have a stomach with four chambers, namely the rumen, reticulum, omasum and abomasum. In the first two chambers, the rumen and the reticulum, the food is mixed with saliva and separates into layers of solid and liquid material. Solids clump together to form the cud, or bolus. The cud is then regurgitated, chewed slowly to completely mix it with saliva, which further breaks down fibers. Fiber, especially cellulose, is broken down into glucose in these chambers by symbiotic bacteria, protozoa and fungi. The broken-down fiber, which is now in the liquid part of the contents, then passes through the rumen into the next stomach chamber, the omasum, where water is removed. The food in the abomasum is digested much like it would be in the human stomach. It is finally sent to the small intestine, where the absorption of the nutrients occurs.

Almost all the glucose produced by the breaking down of cellulose is used by the symbiotic bacteria. Ruminants get their energy from the volatile fatty acids produced by the bacteria, namely lactic acid, propionic acid and butyric acid.

The rumen is the major source of methane production in ruminants.

Examples of ruminants are listed above. However, in at least some embodiments, the bacteria are used as an additive for foodstuffs for domesticated livestock such as cattle, goats, sheep and llamas. In at least some embodiments, the strains and methods provided herein are used in cattle.

By “administer,” is meant the action of introducing at least one strain of bacterium described herein into the animal's gastro-intestinal tract. More particularly, this administration is an administration by oral route. This administration can in particular be carried out by supplementing the feed intended for the animal with the at least one strain of bacterium; the supplemented feed then being ingested by the animal. The administration can also be carried out using a stomach tube or any other way to make it possible to directly introduce the at least one strain of bacterium into the animal's gastro-intestinal tract.

By “effective amount,” is meant a quantity of bacteria sufficient to allow improvement, i.e., reduction in the amount of methane production in comparison with a reference. The methane reductive effect can be measured in the rumen with an artificial rumen system, such as that described in T. Hano (1993) or by in vivo oral administration to ruminants.

This effective amount can be administered to the ruminant animal in one or more doses.

By “at least one strain,” is meant a single strain but also mixtures of strains comprising at least two strains of bacteria.

By “a mixture of at least two strains,” is meant a mixture of two, three, four, five, six or even more strains.

In some embodiments of a mixture of strains, the proportions can vary from 1% to 99%. Other embodiments of a mixture of strains are from 25% to 75%. Additional embodiments of a mixture of strains are approximately 50% for each strain. When a mixture comprises more than two strains, the strains can be present in substantially equal proportions in the mixture.

As used herein, a “variant” has at least 80% identity of genetic sequences with the disclosed strains using random amplified polymorphic DNA polymerase chain reaction (RAPD-PCR) analysis. The degree of identity of genetic sequences can vary. In some embodiments, the variant has at least 85%, 90%, or 95% identity of genetic sequences with the disclosed strains using RAPD-PCR analysis. Six primers that can be used for RAPD-PCR analysis include the following: Primer 1 (5′-GGTGCGGGAA-3′), PRIMER 2 (5′-GTTTCGCTCC-3′), PRIMER 3 (5′-GTAGACCCGT-3′), PRIMER 4 (5′-AAGAGCCCGT-3′), PRIMER 5 (5′-AACGCGCAAC-3′), PRIMER 6 (5′-CCCGTCAGCA-3′). RAPD analysis can be performed using Ready-to-Go™ RAPD Analysis Beads (Amersham Biosciences, Sweden), which are designed as pre-mixed, pre-dispensed reactions for performing RAPD analysis.

In one embodiment of preparing the Propionibacterium strain, the strain is fermented to a 5×10⁸ CFU/ml to a 4×10⁹ CFU/ml level, to a level of 2×10⁹ CFU/ml or to any desired concentration. The bacteria are harvested by centrifugation, and the supernatant is removed. The pelleted bacteria can then be fed to a ruminant. The pelleted bacteria can also be freeze-dried before being administered to a ruminant.

Some embodiments of the methods include a step of administering other microorganisms with the Propionibacterium strain. The microorganisms can be selected from the group comprising in particular the lactic bacteria, probiotic microorganisms, yeasts, and fungi (for example Penicillium and Geotrichum).

In at least some embodiments of the methods, the effective amount of the at least one strain of bacterium is typically comprised between 10⁵ CFU and 10¹³ CFU per animal and per day, particularly between 10⁷ CFU and 10¹² CFU per animal and per day, more particularly between 10⁸ CFU and 10¹¹ CFU per animal and per day, even more particularly approximately 10¹⁰ CFU per animal and per day.

The bacterium provided herein can be administered, for example, as the bacterium-containing culture solution, the bacterium-containing supernatant or the bacterial product of a culture solution.

The bacterium may be administered to the ruminant in one of many ways. For example, the culture can be administered in a solid form as a veterinary pharmaceutical, may be distributed in an excipient, preferably water, and directly fed to the animal, may be physically mixed with feed material in a dry form or the culture may be formed into a solution and thereafter sprayed onto feed material. The method of administration of the culture to the animal is considered to be within the skill of the artisan.

When used in combination with a feed material, the feed material can be grain or hay or silage or grass, or combinations thereof. Included amongst such feed materials are corn, dried grain, alfalfa, any feed ingredients and food or feed industry by-products as well as bio fuel industry by-products and corn meal and mixtures thereof.

The bacterium of the novel process may optionally be admixed with a dry formulation of additives including but not limited to growth substrates, enzymes, sugars, carbohydrates, extracts and growth promoting micro-ingredients. The sugars could include the following: lactose; maltose; dextrose; malto-dextrin; glucose; fructose; mannose; tagatose; sorbose; raffinose; and galactose. The sugars range from 50-95%, either individually or in combination. The extracts could include yeast or dried yeast fermentation solubles ranging from 5-50%. The growth substrates could include: trypticase, ranging from 5-25%; sodium lactate, ranging from 5-30%; and, Tween 80, ranging from 1-5%. The carbohydrates could include mannitol, sorbitol, adonitol and arabitol. The carbohydrates range from 5-50% individually or in combination. The micro-ingredients could include the following: calcium carbonate, ranging from 0.5-5.0%; calcium chloride, ranging from 0.5-5.0%; dipotassium phosphate, ranging from 0.5-5.0%; calcium phosphate, ranging from 0.5-5.0%; manganese proteinate, ranging from 0.25-1.00%; and, manganese, ranging from 0.25-100%.

The time of administration is not crucial so long as the reductive effect on methane production is shown. As long as the feed is retained in the rumen, administration is possible at any time. However, since the bacterium is preferably present in the rumen at about the time methane is produced, the bacterium is preferably administered with or immediately before feed.

Thus, in at least some embodiments, the effective amount of at least one strain of bacterium is administered to a ruminant animal by supplementing a feed intended for the animal with the effective amount of at least one strain of bacterium. As used herein, “supplementing” means the action of incorporating the effective amount of bacteria provided herein directly into the feed intended for the animal. Thus, the animal, when feeding, ingests the bacteria provided herein.

A feed supplement for a ruminant animal comprises at least one strain of Propionibacterium bacterium.

The invention will now be further described by way of Examples, which are meant to serve to assist one of ordinary skill in the art in carrying out the invention and are not intended in any way to limit the scope of the invention.

### EXAMPLES

The following Examples are provided for illustrative purposes only. The Examples are included herein solely to aid in a more complete understanding of the methods, compositions and kits described herein. The Examples do not limit the scope of the invention described or claimed herein in any fashion.

### Example 1

### Propionibacteria Increase Ruminal Propionate

An experiment to determine whether a propionate producer could increase propionate levels in the rumen was tested and described in Stein et. al., 2006. Thirty-eight pregnant dairy cows were assigned to three treatments. One group was designated as control cows (CON, n=13) and did not receive the direct-fed microbial (DFM), Propionibacterium acidipropionici P169. The other two groups received the DFM at a high dose, 6×10¹¹ cfu/head/day (HI, n=11) or a low dose, 6×10¹⁰ cfu/head/day (LO, n=14). The cows were on treatment from −2 weeks prepartum until 30 weeks postpartum. Rumen fluid was collected from all cows for analysis of pH and short-chain fatty acids at 60, 120, and 175 days in milk (DIM). Ruminal acetate levels were not significantly different between treatment groups. Ruminal propionate levels were influenced (P<0.05) by treatment such that HI cows averaged 18.5 and 17.1% greater than LO and CON, respectively over +60, +120 and +175 DIM. The HI cows averaged 15.5% and 13.3% lower A:P levels (P<0.06) compared to the LO and CON cows, respectively. The pH was influenced by treatment (P<0.02) such that over +60, +120 and +175 DIM, P169 cows had lower pH in comparison to the LO and CON with a pH of 6.65 (±0.07) compared to 6.94 (±0.06) and 6.86 (±0.06).

The results of this published study demonstrate the ability of P. acidipropionici P169 to increase propionate levels in the rumen when fed at a high dose.

### Example 2

### Propionibacteria Increase Ruminal Propionate

A trial was conducted to determine the effect of feeding a yeast product alone or in combination with Propionibacterium acidipropionici P169, to determine the effects on rumen parameters. The trial is described in Lehloenya et. al., 2008. Control (CON) cattle received the basal ration. Treated cattle received the basal ration plus one of three treatments, P. acidipropionici P169 (P169 or DFM) fed at 6×10¹¹ CFU/head/d, Diamond V-XP yeast (XPY) fed at 56 g/head/d, and Diamond V-XP fed at 56 g/head/d plus P. acidipropionici P169 fed at 6×10¹¹ CFU/head/d (XPY+P169). DFM and yeast treatments were applied at a rate of 4.5 kg of TMR once daily and fed to steers individually for 21 day study periods. Days 1-15 were used for adaptation and days 16-21 for sample collection. Ruminal kinetics were measured on days 19 and 20 by pulse dosing a solution containing cobalt-EDTA and collecting rumen fluid 6× over the next 24 hours. Rumen NH3—N and pH were not affected by treatment. Total VFA concentration was not affected by treatment; however, P169 increased (P=0.05) ruminal propionate by 9.5% above steers not receiving P169 and tended (P=0.06) to decrease ruminal acetate. This resulted in a tendency for a lower ratio of acetate:propionate (P=0.06; 10.7%) for steers fed P169.

The results of this published study demonstrate the ability of P. acidipropionici P169 to influence ruminal VFA levels of propionate and acetate.

### Example 3

### In Vitro Methane Measurements

Two rumen fluid in vitro experiments were performed to determine if Propionibacteria reduced methane accumulation in buffered rumen fluid supplemented with 3% dextrose and inoculated with Propionibacterium strains compared to an uninoculated control after 4 and 8 hours.

Rumen In Vitro Protocol

### Rumen Fluid Collection.

Thermos® flasks were warmed with hot water prior to collection. Six Thermos® flasks were filled with cheesecloth-filtered rumen fluid, i.e., three flasks per cow. Each flask was filled three quarters full and then capped off with fresh digesta to reduce oxygen diffusing into the rumen fluid. Digesta was discarded.

### Rumen Fluid Processing in the Lab.

A sterile 2 liter Pyrex® bottle was flushed with CO₂ for two minutes. A funnel was placed in the bottle together with a CO₂ tube. The tube was not immersed in rumen fluid. The bottle was placed in a sink, and the sink was filled with warm water. Two layers of cheesecloth were placed over the funnel. The Thermos® flask was vented by opening its flap. The Thermos® flask was inverted and shook to pour rumen fluid out of a small opening in the Thermos® flask. One Thermos® flask from each animal was emptied into the Pyrex® bottle. The Pyrex® bottle was flushed with CO₂ and capped tightly. This was repeated with rumen fluid in remaining Thermos® flasks.

### Sample Preparation.

Sterile 250 ml Pyrex bottles to which 36 ml sterile buffer and 5.4 g dextrose was added were warmed in a shaking water bath. Cheesecloth filtered rumen fluid (144 ml), collected from dairy cows, was added to each bottle. The bacterial cultures were added at time 0. At time 0, 4 hours and 8 hours methane emissions were measured with a X-am 7000 (Draeger).

### Experimental Design.

Two bottles per water bath served as an uninoculated control with glucose, but no culture, i.e., no DFM. Duplicates of each treatment were in separate water baths, i.e., there were four bottles per treatment. Samples were collected at time 0 hour, 4 hours and 8 hours. Caps were opened carefully to release accumulated gases.

Peak methane emission will be measured by X-am 7000 (Draeger). Samples were collected by dispensing about 7 mL of the sample into a Falcon tube. Bottles were flushed with CO₂ during additions and sample collection. The sample was stored in the freezer for VFA analysis by HPLC.

Treatments Experiment One: uninoculated control (DFM-); P5; P54; P63; P169; and P195.

Treatments Experiment Two: uninoculated control (DFM-); P25; P49; P 104; P170; and P179.

### Merten's Buffer for In Vitro Rumen Digestion

In Vitro Buffer Solution
1.0 L Distilled Water
4.0 g Ammonium Bicarbonate
35.0 g Sodium Bicarbonate
Total of 1.0 Liter

### Statistical Analysis

Analysis of variance was performed using standard least squares in JMP 5.0.1a (SAS Institute, Inc., Cary, N.C.). The model was a completely randomized design with each time point analyzed individually. The model included the effect of DFM supplementation. Separation of means was performed by student's T test. The null hypothesis was that DFM supplementation would have no effect on methane accumulation relative to controls devoid of DFM. Differences between treatments were regarded as statistically significant at P≦0.05.

Results

In experiment one, there was a numerical reduction of methane for all propionibacteria at both time points (Table 1). The difference was significant for P5 compared to the uninoculated control after 4 hours, but not at 8 hours. At 8 hours the differences were significant for P195, P54 and P63.

In experiment two, there was a significant reduction in methane accumulation for all propionibacteria strains at 4 hours (Table 2). The reduction was still significant at 8 hours for strains P170, P179 and P25.

In experiment one, methane accumulated over the 8 hours of the experiment. However, in experiment two methane levels decreased after 8 hours, possibly indicating that methanogens had not survived the lowering of the pH caused by acid production as the dextrose was fermented.

This data indicates that Propionibacterium strains can reduce methane accumulation in rumen fluid. Results of the in vitro studies of these strains will reasonably correlate with methods of using one or more of these strains in vivo to reduce methane production in ruminants.

TABLE 1
Methane (% vol) accumulation in the first experiment in buffered
rumen fluid supplemented with dextrose with Propionibacteria or
devoid of DFM (DFM−) (4 h P = 0.3266, 8 h P = 0.1262).

| Experiment 1 | 4 h | | | 8 h | | |
|---|---|---|---|---|---|---|
| Treatment | LSMeans | | SEM | LSMeans | | SEM |
| DFM− | 2.27 | A | 0.22 | 3.22 | A | 0.31 |
| P169 | 1.81 | AB | 0.22 | 2.48 | AB | 0.31 |
| P195 | 1.87 | AB | 0.22 | 2.24 | B | 0.31 |
| P5 | 1.52 | B | 0.22 | 2.34 | AB | 0.31 |
| P54 | 1.80 | AB | 0.22 | 2.18 | B | 0.31 |
| P63 | 1.82 | AB | 0.22 | 1.96 | B | 0.31 |

^{A,B}Means without common superscripts are significantly different (P ≦ 0.05) within time-points.

TABLE 2
Methane (% vol) accumulation in the second experiment in buffered
rumen fluid supplemented with dextrose with propionibacteria or
devoid of DFM (DFM−) (4 h P < 0.0001, 8 h P = 0.0081).

| Trial 2 | 4 h | | | 8 h | | |
|---|---|---|---|---|---|---|
| Treatment | LSMeans | | SEM | LSMeans | | SEM |
| DFM− | 2.43 | A | 0.14 | 1.64 | A | 0.19 |
| P104 | 1.13 | B | 0.14 | 1.21 | AB | 0.19 |
| P170 | 1.38 | B | 0.14 | 0.93 | B | 0.19 |
| P179 | 1.30 | B | 0.14 | 1.05 | B | 0.19 |
| P25 | 1.28 | B | 0.14 | 1.06 | B | 0.19 |
| P49 | 1.43 | B | 0.14 | 1.11 | AB | 0.19 |

^{A,B,C}Means without common superscripts are significantly different (P ≦ 0.05) within time-points.

### Example 4

### Volatile Fatty Acid Analysis

Volatile fatty acid concentrations will be determined by HPLC analysis using procedures known in the art, such as the following. Samples will be thawed on ice. Samples will be centrifuged at 5000 rpm for 10 mM. Supernatant will be transferred into a syringe attached to 0.2 μL filter. Supernatant will be filtered into an HPLC vial. HPLC vials will be placed in the HPLC carrousels. A vial of rumen fluid standard (e.g., 0.1% standard) will be placed in the first slot in carrousel A. Samples will be run on Rumen 2011 HPLC method with using the Aminex HPX-87H column (Bio-Rad) with 5 mM sulfuric acid mobile phase at 0.8 mL per minute at 60C.

### Example 5

### Animal Studies

Twelve cattle of approximately 4 months of age will be used. Cattle will be randomized by treatment (e.g., 4 cattle/treatment).

There will be four treatments with the same number of animals per treatment. The four treatments will consist of balanced diets containing the following: Diet 1: Control; Diet 2: propionibacteria strain 1; Diet 3: propionibacteria strain 2; Diet 4: propionibacteria strain 3. Where one or more propionibacteria strain is included, it/they will be administered in an effective amount. The effective amount of the at least one strain of bacterium is typically comprised between 10⁵ CFU and 10¹³ CFU per animal and per day.

Diet Digestibility: Cattle will be housed in individual pens in a metabolism facility during a 14-day adaptation period with ad libitum access to food, which will be delivered manually once daily, and water. On day 15, the cattle will be fitted with a fecal collection bag and moved to a metabolic crate in the Controlled Environment Building to allow total collection of feces and urine. CH₄ and CO₂ emissions will be measured as well. The cattle will be housed in the metabolic crates for 4 days, and will have 90% ad libitum access to feed and ad libitum access to water. During these 4 days, the total weight of feed, orts, feces and urine will be recorded. On days 15-18, 10% sub samples will be collected from feed, orts, faeces and urine for analysis before morning feeding. These samples will be analyzed for crude protein, (CP) nitrogen (N), acid detergent insoluble nitrogen (ADIN), gross energy (GE), neutral detergent fibre (NDF), and acid detergent fibre (ADF), ether extract (EE), dry matter (DM), organic matter (OM) and ash, starch (non-fibre carbohydrates, NFC), calcium (Ca) and phosphorous (P).

Methane measurements: The methane measurements will be done using the environmental chamber technique during the same 4 day period of the digestibility trial. For these measurements, cattle will be assigned to an environmental chamber (4.4 m wide×3.7 m deep×3.9 m tall). The total quantity of CH₄ emitted in the chambers will be quantified by measuring the gradient of influx and exhausted concentrations and volumes. The air volume in each chamber will be exchanged every 5 min. Methane concentration will be recorded every 30 min by a calibrated infrared gas analyzer. The daily CH₄ flux will be expressed per unit of DMI of the animals within the chambers, because CH₄ emission is known to be a function of the DMI.

Animal Chamber Experiments and Animal Performance Studies

The propionibacteria showing the most CH₄ reduction will be used again in an additional chamber experiment as described above. An animal performance study will be conducted.

A study will be conducted to determine growth performance using 160 crossbred beef steers (350 kg) allocated to 4 feedlot finishing diets (4 pens/treatment, 10 steers/pen). The experiment will be a completely randomized design with a 2×2 factorial arrangement of treatments. Randomization will be stratified to ensure a uniform weight across the pens. The steers will be housed in outdoor feedlot pens. The treatments (diets) will contain varying levels of inclusion of the propionibacteria strain that showed the greatest potential for methane reduction as determined by previous studies. Where one or more propionibacteria strain is included, it/they will administered in an effective amount. The effective amount of the at least one strain of bacterium is typically comprised between 10⁵ CFU and 10¹³ CFU per animal and per day.

All diets will be formulated to meet or exceed nutrient requirements of beef cattle (NRC, 1996). Monensin® antibiotic will be included in all diets at a concentration of 25 ppm.

The steers will be purchased from local auction markets and processed upon arrival with follow commercial practices (i.e., ear tagging, branding) and vaccinating against IBR, PI3 and Haemophilus somnus (Resvac 2/Somubac, Pfizer Animal Health) and against Clostridium spp. (Tasvax 8, Schering-Plough Animal Health, Upper Hutt, NZ) as well as implants. The steers will be adapted to a barley or wheat-based finishing diets for 4 wk prior to commencing the study. Two pens from each treatment will be equipped with the GrowSafe System (GrowSafe Systems Ltd., Airdrie, AB Canada), which will monitor and record the feeding behavior of individual animals including frequency and duration of visits to the feed bunk, meal size, eating rates and individual feed intake. The steers will be weighed individually on two consecutive days at the start and at the end of the trial, and at 28-d intervals. The steers will be fed total mixed rations once daily. Refusals will be weighed weekly and sampled on weigh day for chemical analysis. Intake, daily gain, and feed efficiency will be determined within each weigh period and the entire trial.

Blood samples (max. 10 ml) will be taken from the jugular vein on d 1, 56, and 112 (or the last weighing before shipping for slaughtering). Measurement will include blood glucose, urea N, triglycerides, NEFA.

All steers will be slaughtered commercially at the end of finishing period. Carcass measurements (weight, backfat, grade, ribeye area, marbling score and liver abscess score) will be conducted. Data will be analyzed using a mixed model that includes the fixed effects of main factors and their interaction. Pen will be a random effect. The repeated statement will be included in the model for overall data analysis. For the data of frequency and duration of visits to the feed bunk, meal size, eating rates and individual feed intake, individual animals will be the unit of analysis.

It is expected that animals receiving the Propionibacteria will emit less methane than the animals not receiving the Propionibacteria.

### Example 6

### Methane Measurements Resulting from Treatment of Simulated Rumen Systems with Selected Direct-Fed Microbial Bacteria

The procedures used in this continuous culture study were similar to those described by Eun et al. (2004). Whole ruminal contents were collected from ruminally cannulated, lactating Holstein cows fed a total mixed ration diet. Ruminal fluid from these animals was placed into fermentor vessels that allowed continuous, independent flow of liquid and particulate matter in the simulated rumen fermentor vessel. Anaerobic conditions in the continuously cultured simulated rumens were maintained by sealing the fermentor openings with rubber and providing a continuous flow of CO₂ to maintain a positive internal pressure. Additionally, artificial saliva was delivered to each ruminal culture. The temperature of the cultures was maintained at the normal bovine internal body temperature. Ruminal contents were stirred continuously.

Feed samples were added to the fermentors twice daily. The direct-fed microbial treatment was administered to the fermentor each time feed was added. The formulation of the feed added to the fermentors is provided in Table 3. Two treatments were tested in the simulated rumen fermentors and included the following: (1) control treatment administering only the common feed ration sample; and (2) Propionibacterium acidipropionici strain P169 with the common feed ration sample.

Four independent runs were operated for each treatment. Each run consisted of two fermentors, with each one being administered one of the two treatments. Each run lasted 10 days, including a 7 day stabilization and adaptation period. This period allowed the simulated rumen fermentors to stabilize in response to the twice daily administration of the feed ration sample and microbial treatment. The 7 day stabilization and adaptation period was followed by a 3 day sample collection period.

Samples obtained in the 3-day sample collection period were pooled for analysis, generating data representative of each treated fermentor in each of the four independent runs. Daily methane production was measured on the samples collected from the continuously cultured simulated rumen fermentors. Daily methane production was reduced (P<0.05) in the rumen fermentation cultures treated with Propionibacterium strain P169 compared to the control cultures (Table 4).

TABLE 3
Ingredients and chemical composition of lactation dairy diet
offered to the continuous cultures^{a}.

| | g/kg DM |
|---|---|
| Ingredient (g/kg DM) | |
| Alfalfa hay | 250 |
| Corn silage | 250 |
| Corn grain, steam flaked | 267 |
| Cottonseed, whole | 73.0 |
| Soybean meal, expeller | 60.4 |
| Soybean hull | 77.4 |
| Vitamin and trace minerals^{b} | 21.8 |
| Nutrient (g/kg DM) | |
| Dry matter (g/kg) | 630 ± 7.8 |
| Organic matter | 930 ± 6.1 |
| Crude protein | 153 ± 2.0 |
| Acid detergent fiber | 262 ± 7.9 |
| Neutral detergent fiber | 363 ± 8.4 |
| Ether extract | 36.2 ± 2.61 |
| Fatty acid (g/100 g FA methyl | |
| esters) | |
| C16:0 | 20.9 ± 0.62 |
| C18:0 | 2.87 ± 0.121 |
| C18:1 cis-9 | 16.8 ± 0.82 |
| C18:1 cis-11 | 1.52 ± 0.081 |
| C18:2 | 41.9 ± 1.18 |
| C18:3 n-3 | 0.84 ± 0.513 |
| C20:0 | 0.44 ± 0.099 |

^{a}Samples pooled by independent run (n = 4).
^{b}Formulated to contain (per kg DM): 4.75 mg of Se (from sodium selenate), 182 mg of Cu (from copper sulfate), 732 mg of Zn (from zinc sulfate), 10,369 IU of vitamin A, 1185 IU of vitamin D, 150 IU of vitamin E, and 19.6 g of Rumensin ® (Elanco Animal Health, Greenfield, IN, USA).

TABLE 4
Production of CH₄ in response to administering a lactation
dairy diet with Propionibacterium acidipropionici strain P169
to continuously cultured simulated rumen fermentors.

Dietary treatment^{a}

| | CON | P169 | SEM | P |
|---|---|---|---|---|
| CH₄ production | 3.24 | 2.07 | 1.266 | <0.05 |
| mM/d | | | | |

^{a}CON: control diet without DFM supplementation; P169: CON supplemented with Propionibacterium acidipropionici strain P169.

It is understood that the various preferred embodiments are shown and described above to illustrate different possible features of the invention and the varying ways in which these features may be combined. Apart from combining the different features of the above embodiments in varying ways, other modifications are also considered to be within the scope of the invention. The invention encompasses all alternate embodiments that fall literally or equivalently within the scope of these claims. Therefore, it is intended that this invention be limited only by the claims and the equivalents thereof. The disclosures of patents, references and publications cited in the application are incorporated by reference in their entirety herein.

### BIBLIOGRAPHY

T. Hano (1993) J. Gen. Appl. Microbiol., 39, 35-45.

Lehloenya, K. V., Krehbiel, C. R., Mertz, K. J., Rehberger, T. G., Spicer, L. J. (2008) Effects of propionibacteria and yeast culture fed to steers on nutrient intake and site and extent of digestion. V 92 (2):653.

Power, E. G., “RAPD typing in microbiology—a technical review,” J. Hosp. Infect. (1996) 34(4):247-265.

Stein, D. R., Allen, D. T., Perry, E. B., Bruner, J. C., Gates, K. W., Rehberger, T. G., Mertz, K. J., Jones, D., Spicer, L. J. (2006) Effects of feeding propionibacteria to dairy cows on milk yield, milk components, and reproduction. J Dairy Sci 89 (1):111.

Thorpe, A. (2009) Enteric fermentation and ruminant eructation: the role (and control?) of methane in the climate change debate. Climatic Change 93 (3):407.

Eun, J. S., V. Fellner, and M. L. Gumpertz. 2004. Methane production by mixed ruminal cultures incubated in dual-flow fermentors. J. Dairy Sci. 87:112-121.

What is claimed is:

1. A method for reducing methane production in a ruminant animal comprising administering to the ruminant animal an effective amount of at least one strain of bacterium of the genus Propionibacterium.

2. The method of claim 1, wherein the strain of bacterium belongs to the species Propionibacterium jensenii, Propionibacterium acidipropionici, Propionibacterium freudenreichii or Propionibacterium freudenreichii ssp shermanii.

3. The method of claim 2, wherein the strain belongs to the species Propionibacterium freudenreichii or Propionibacterium freudenreichii ssp shermanii.

4. The method of claim 2, wherein the strain belongs to the species Propionibacterium jensenii.

5. The method of claim 2, wherein the strain belongs to the species Propionibacterium acidipropionici.

6. The method of claim 2, wherein the strain is selected from the group consisting of P169, P170, P179, P195, P261, P5, P63, P54, P25, P49, P104, strains having all the characteristics thereof, any derivative or variant thereof, and mixtures thereof.

7. The method of claim 2, wherein the strain is selected from the group consisting of P169, P170, P179, P195, P261, P5, P63, P54, P25, P49, P104, any derivative or variant thereof, and mixtures thereof.

8. The method of claim 1, wherein the effective amount of at least one strain of bacterium is administered to the ruminant animal by supplementing food intended for the animal with the effective amount of at least one strain of bacterium.

9. The method of claim 1, wherein the ruminant animal is selected from the members of the Ruminantia and Tylopoda suborders.

10. The method of claim 1, wherein the ruminant animal is selected from the members of the Antilocapridae, Bovidae, Cervidae, Giraffidae, Moschidae, Tragulidae families.

11. The method of claim 10, wherein the ruminant animal is a cattle, goat, sheep, giraffe, bison, yak, water buffalo, deer, camel, alpaca, llama, wildebeest, antelope, pronghorn or nilgai.

12. A feed supplement for a ruminant animal for reducing methane production comprising at least one strain of bacterium of the genus Propionibacterium.

13. The feed supplement of claim 12, comprising at least one strain of bacterium belonging to the species Propionibacterium jensenii, Propionibacterium acidipropionici, Propionibacterium freudenreichii and Propionibacterium freudenreichii ssp shermanii.

14. The feed supplement of claim 13, wherein the strain belongs to the species Propionibacterium freudenreichii or Propionibacterium freudenreichii ssp shermanii.

15. The feed supplement of claim 13, wherein the strain belongs to the species Propionibacterium jensenii.

16. The feed supplement of claim 13, wherein the strain belongs to the species Propionibacterium acidipropionici.

17. The feed supplement of claim 13, wherein the strain is selected from the group consisting of P169, P170, P179, P195, P261, P5, P63, P54, P25, P49, P104, strains having all the characteristics thereof, any derivative or variant thereof, and mixtures thereof.

18. The feed supplement of claim 13, wherein the strain is selected from the group consisting of P169, P170, P179, P195, P261, P5, P63, P54, P25, P49, P104, any derivative or variant thereof, and mixtures thereof.

19. A method for reducing methane production by a ruminant animal comprising administering to the animal a feed supplement comprising an effective amount of at least one strain of bacterium of the genus Propionibacterium.

20. The method of claim 19, further comprising increasing feed efficiency or propionate production in the ruminant.
